(19) United States
(12) Patent Application Publication
(10) Pub. No.: US 2023/0074506 A1
(43) Pub. Date: Mar. 9, 2023

(54) PROCESS FOR RECOVERING & PURIFYING HUMAN MILK OLIGOSACCHARIDES

(71) Applicant: DSM IP ASSETS B.V., HEERLEN (NL)

(72) Inventors: Arthur Maurits Christiaan JANSE, Columbia, MD (US); Pierre CHASSAGNE, Columbia, MD (US)

(21) Appl. No.: 17/759,590

(22) PCT Filed: Jan. 29, 2021
(86) PCT No.: PCT/US2021/015722

§ 371 Date: Jul. 27, 2022

Related U.S. Application Data
(60) Provisional application No. 62/967,357, filed on Jan. 29, 2020.

(51) Int. Cl. C12P 19/04; C07H 3/06; C07H 1/08

(52) U.S. Cl. CPC C12P 19/04; C07H 3/06; C07H 1/08

(57) ABSTRACT

A process for recovery and purification of HMOs comprising: (a) providing an HMO-containing fermentation broth comprising biomass; (b) separating the fermentation broth to form a separated HMO-containing stream and a biomass waste stream; (c) purifying the separated HMO-containing stream; (d) concentrating the separated HMO-containing stream; and (e) drying the product of steps (a)-(d) by an indirect drying method thereby forming a purified HMO, wherein steps (c)-(d) can be performed in any order.

### CROSS REFERENCE TO RELATED APPLICATIONS

This application is an International Application claiming priority to U.S. Provisional Application No. 62/967,357, filed 29 Jan. 2020, the entire contents of which are hereby incorporated by reference in their entirety.

### FIELD OF THE INVENTION

The invention relates to separation processes. More particularly, the invention relates to processes for recovering and purifying human milk oligosaccharides (HMOs) from a fermented broth.

### BACKGROUND OF THE INVENTION

Human milk contains a family of unique oligosaccharides, HMOs, which are structurally diverse unconjugated glycans. Despite being the third most abundant solid component of human milk (after lactose and fat), human infants cannot actually digest HMOs. Instead, they function as prebiotics to help establish commensal bacteria. HMOs also function as anti-adhesives that help prevent the attachment of microbial pathogens to mucosal surfaces. Supporting the development of a healthy digestive tract in infants assists in the development of their immune systems since much of the infant's immune system is in the digestive tract. The occurrence and concentration of these complex oligosaccharides are specific to humans and are not found in large quantities in the milk of other mammals such as domesticated dairy animals. There is thus a need for HMO-containing supplements for use with formula fed to infants that provides these beneficial effects. Work has been ongoing to develop improved systems for producing HMOs, as in EP 14827224.8, WO2019/003133, WO2019/003135, U.S. 2017/0304375, and WO2019/003136. In particular, these processes often make use of a spray-drying step in the recovery process, e.g., as shown in WO2019/110801, WO2019/110806, WO2015/106943, WO2019/110804 and WO2019/110800. Nevertheless, there remains a need to provide improved processes for the production and recovery of HMOs relative to conventional processes.

### SUMMARY OF THE INVENTION

The subject matter of the present disclosure includes processes for recovering and purifying HMOs from a fermentation broth. It has unexpectedly been discovered that by recovering, purifying, and drying the HMO in a particular manner, improvements in operational safety and reduced product losses are possible.

In one embodiment, the present disclosure provides a process for recovery and purification of HMOs comprising: (a) providing an HMO-containing fermentation broth comprising biomass; (b) separating the fermentation broth to form a separated HMO-containing stream and a biomass waste stream; (c) purifying the separated HMO-containing stream; (d) concentrating the separated HMO-containing stream; and (e) drying the product of steps (a)-(d) by an indirect drying method thereby forming a purified HMO, wherein steps (c)-(d) can be performed in any order. Purification step (c) can be selected from at least one of: (i) ultrafiltration; (ii) nanofiltration; (iii) deionization treatment; and (iv) decolorization, wherein sub-steps i-iv can be performed in any order. Deionization treatment step (iii) can be selected from ion adsorption or ion exchange.

In another embodiment, the present disclosure provides a process for recovery and purification of HMOs comprising: (a) providing an HMO-containing fermentation broth comprising biomass; (b) separating the fermentation broth to form a separated HMO-containing stream and a biomass waste stream; (c) purifying the separated HMO-containing stream in a step selected from at least one of ultrafiltration, nanofiltration, ion adsorption and decolorization, performed in any order; (d) concentrating the separated HMO-containing stream; and (e) drying the product of steps (a)-(d) by an indirect drying method thereby forming a purified HMO, wherein steps (c)-(d) can be performed in any order.

In another embodiment, the present disclosure provides a process for recovery and purification of HMOs comprising: (a) providing an HMO-containing fermentation broth comprising biomass; (b) separating the fermentation broth to form a separated HMO-containing stream and a biomass waste stream; (c) purifying the separated HMO-containing stream in a step selected from at least one of ultrafiltration, nanofiltration, ion exchange treatment and decolorization, performed in any order; (d) concentrating the separated HMO-containing stream; and (e) drying the product of steps (a)-(d) by an indirect drying method thereby forming a purified HMO, wherein steps (c)-(d) can be performed in any order.

In another embodiment, the present disclosure further provides a method comprising drying a purified HMO stream having a dry matter content of 20 to 80 wt % with an indirect drying method, thereby forming a dried HMO product having a moisture level of no more than 9 wt. %.

In still another alternate embodiment, the present disclosure further provides a dried HMO product produced by the above process having a monosaccharide content of less than 9%, measured via high performance anion-exchange chromatography coupled with pulsed amperometric detection (HPAEC-PAD).

In another embodiment, the present disclosure further provides a dried HMO produced by the above process having a color absorption in solution of less than 0.3, measured according to the method described below.

In another embodiment, the present disclosure provides a process comprising: providing an HMO-containing fermentation broth comprising biomass; removing the biomass thereby forming a biomass-depleted stream; purifying the biomass-depleted stream, thereby forming a purified HMO stream containing 20-80 wt. % solids and 20-80 wt. % liquid; and drying the purified HMO stream by an indirect drying method to form an HMO solids stream containing at least 90 wt. % solids.

In one embodiment, the present disclosure provides a process comprising: (a) providing an HMO-containing fermentation broth comprising biomass; (b) centrifuging the fermentation broth to form a biomass-enriched stream and a biomass-depleted product stream; (c) filtering the biomass-depleted product stream by microfiltration to form a low-suspended matter product stream; (d) filtering the low-suspended matter product stream by ultrafiltration to form an ultrafiltration product stream; (e) filtering the ultrafiltration product stream by nanofiltration to form a nanofiltration product stream; (f) subjecting the nanofiltration product stream to cation exchange, thereby forming a cation-depleted product stream; (g) decolorizing the cation-depleted product stream, thereby forming a decolorized product stream; (h) subjecting the decolorized product stream to anion exchange, thereby forming an anion-depleted product stream; (i) concentrating the anion-depleted product stream, thereby forming a concentrated product stream; and (j) drying the concentrated product stream with an indirect drying method to form an HMO-enriched product, wherein the product of step (i) is optionally subjected to a heat treatment step between steps (i) and (j).

In an alternate embodiment, the present disclosure provides a process comprising: (a) providing an HMO-containing fermentation broth comprising biomass; (b) centrifuging the fermentation broth to form a biomass-enriched stream and a biomass-depleted product stream; (c) filtering the biomass-depleted product stream by ultrafiltration to form an ultrafiltration product stream; (d) filtering the ultrafiltration product stream by nanofiltration, thereby forming a nanofiltration product stream; (e) subjecting the nanofiltration product stream to cation exchange, thereby forming a cation-depleted product stream; (f) decolorizing the cation-depleted product stream, thereby forming a decolorized product stream; (g) subjecting the decolorized product stream to anion exchange, thereby forming an anion-depleted product stream; (h) concentrating the anion-depleted product stream, thereby forming a concentrated product stream; and (i) drying the concentrated product stream with an indirect drying method to form an HMO-enriched product, wherein the product of step (h) is optionally subjected to a heat treatment step between steps (h) and (i).

In another embodiment, the present disclosure provides a process for the production of HMOs comprising nanofiltration, ion-exchange or ion adsorption, and concentration by evaporation, in any order, followed by indirect drying.

In still another embodiment, the present disclosure provides an HMO produced by indirect drying comprising at least one of (i) <2% lactulose; (ii) <3% fucose; (iii) <1% galactose; or (iv) <3% glucose.

In another embodiment, the present disclosure provides a process for the production of HMOs comprising drying an HMO-containing stream in a drum dryer, the dryer comprising chrome-plated surfaces contacting the HMO-containing stream.

In still another embodiment, the present disclosure provides an HMO produced by indirect drying comprising <5 wt. % water.

In still another embodiment, the present disclosure provides an HMO produced by indirect drying comprising fines fraction less than 10%, preferably less than 5%, more preferably less than 1%, most preferably less than 0.1%.

### DETAILED DESCRIPTION OF THE INVENTION

The present disclosure features methods for recovering and purifying human milk oligosaccharides (HMOs) comprising one or more of the following process steps: fermentation of a genetically modified microbial organism; centrifugation or filtration, and microfiltration to remove biomass (e.g., cells, high molecular weight molecules); ultrafiltration to remove proteins and/or other higher molecular weight molecules such as DNA; a nanofiltration step to remove molecules that are smaller than the desired HMO; decolorization to remove color materials; ion exchange to remove charged molecules, and concentration to remove liquids. In all cases, indirect drying is used to produce HMO products.

### HMO

A desired HMO, such as 2′-fucosyllactose (2′-FL), is purified from a fermentation broth that is a product of the fermentation. After fermentation, the broth containing the desired HMO is applied to the separation process as summarized below.

The term “fermentation broth”, as used in this specification, refers to the product obtained from fermentation of the microbial organism. Thus, the fermentation product comprises cells (biomass), the fermentation medium, residual substrate material, and any molecules/by-products produced during fermentation, such as the desired HMO. After each step of the purification method, one or more of the components of the fermentation product is removed, resulting in a more purified HMO.

Preferably, the desired HMO purified according to the methods of the present disclosure is selected from: 2′-fucosyllactose, 3-fucosyllactose, 2′,3- difucosyllactose, lacto-N-triose II, lacto-N-tetraose, lacto-N-neotetraose, lacto-N-fucopentaose I, lacto-N-neofucopentaose, lacto-N-fucopentaose II, lacto-N-fucopentaose Ill, lacto-N-fucopentaose V, lacto-N-neofucopentaose V, lacto-N-difucohexaose I, lacto-N-difucohexaose II, 6′-galactosyllactose, 3-galactosyllactose, lacto-N-hexaose and lacto-N-neohexaose, sialyl-lacto-N-tetraose a, sialyllacto-N-tetraose b, sialyllacto-N-tetraose c, disialyllacto-N-tetraose, 3′ and 6′ sialyllactose, or mixtures thereof. More preferably, the desired HMO is 2′-fucosyllactose.

The desired HMO, such as 2′-FL, is produced by fermentation of a genetically modified microbial organism. Fermentation may be performed in any suitable fermentation medium, such as, for example, a chemically defined fermentation medium. The fermentation medium may vary based on the microbial organism used.

Preferably, the microbial organism is a genetically modified yeast such as a Saccharomyces strain, a Candida strain, a Hansenula strain, a Kluyveromyces strain, a Pichia strain, a Schizosaccharomyces stain, a Schwanniomyces strain, a Torulaspora strain, a Yarrowia strain, or a Zygosaccharomyces strain. More preferably, the yeast is Saccharomyces cerevisiae, Hansenula polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbruecki, Yarrowia hpolytica, Zygosaccharomyces rouxii, or Zygosaccharomyces baili.

Preferably, the microbial organism can also be selected from the genera E. coli or S. cerevisiae, Bifidobacterium, Lactobacillus, Enterococcus, Strepto coccus, Staphylococcus, Peptostreptococcus, Leuconostoc, Clostridium, Eubacterium, Veilonella, Fusobacterium, Bacterioides, Prevotella, Escherichia, Propionibacterium and Saccharomyces, Bifidobacterium adolescentis, B. animalis, B. bifidum, B. breve, B. infantis, B. lactis, B. longum; Enterococcus faecium; Escherichia coli, Klyveromyces marxianus; Lactobacillus acidophilus, L. bulgaricus, L. casei, L. crispatus, L. fermentum, L. gasseri, L. helveticus, L. johnsonii, L. paracasei, L. plantarum, L. reuteri, L. rhamnosus, L. salivarius, L. sakel, Lactococcus lactis (including but not limited to the subspecies lactis, cremoris and diacetylactis); Leuconostoc mesenteroides (including but not limited to subspecies mesenteroides); Pedicoccus acidilactici, P. pentosaceus; Propionibacterium acidipropionici, P. freudenreichii ssp. shermanii; Staphylococcus carnosus; and Streptococcus thermophilus.

The various unit operations utilized in the processes described in the present disclosure are described below. Combinations of these unit operations can be utilized depending upon the nature of the target HMO and desired product purity. In each case, however, indirect drying is utilized to reduce the moisture level to its target level following the recovery/purification steps.

### Conventional Filtration

For the purpose of this specification, the term conventional filtration refers to processes using plate and frame filtration, recessed chamber filtration, belt filtration, vacuum filtration, horizontal metal leaf filtration, vertical metal-leaf filtration, stacked-disc filtration, rotary vacuum filtration and combinations thereof.

### Centrifugation

Centrifugation can be used to remove suspended matter such as biomass. The desired HMO product is contained in the liquid not retained by the centrifuge. Preferably, the centrifuge is operated continuously.

### Microfiltration

A cross flow filtration process can be used to serve as a guard filter to remove residual biomass that is not removed in the centrifugation step. Typically, the microfiltration has a cut off of 10 microns, preferably a cut off of 2 microns; even more preferably the microfiltration has a cut off of 0.2 to 2.0 microns, and most preferably the microfiltration has a cut off of 0.2 to 0.5 micron. The product stream is the liquid permeate.

### Ultrafiltration

A cross flow ultrafiltration can be used to remove proteins and other high molecular weight compounds, such as DNA and large polysaccharides from the fermentation product. The pore size of the ultrafiltration membrane ranges from about a 300 kD molecular weight cut-off (“MWCO”) or less to about 1 kD MWCO. The product stream is the permeate.

Preferably, the yield of the desired HMO in the permeate after an ultrafiltration step is greater than 50%, greater than 60%, greater than 70%, greater than 80%, greater than 90%, greater than 91%, greater than 92%, greater than 93%, greater than 94%, greater than 95%, greater than 96%, greater than 97%, greater than 98%, or greater than 99%.

### Nanofiltration

Cross flow nanofiltration can be used to remove low molecular weight molecules smaller than the desired HMO, such as mono- and disaccharides, peptides, small organic acids, water, and salts. The pore size of the nanofiltration membrane is fin about 1000 dalton (Da) or less molecular weight cut-off to 200 Da MWCO or less. Preferably, 500 dalton (Da) or less molecular weight cut-off, 450 Da MWCO, 400 Da MWCO, 350 Da MWCO, 300 Da MWCO, 250 Da MWCO, or 200 Da MWCO or less. The product stream is the retentate.

Preferably, the yield of the desired HMO in the retentate after a nanofiltration step is greater than 50%, greater than 60%, greater than 70%, greater than 80%, greater than 90%, greater than 91%, greater than 92%, greater than 93%, greater than 94%, greater than 95%, greater than 96%, greater than 97%, greater than 98%, or greater than 99%.

### Deionization Treatment

A deionization treatment can be used to separate charged molecules from the HMO-containing stream. The deionization treatment can include an ion exchange treatment, ion adsorption or both, using synthetic resins. Such synthetic resins can include cation exchangers, anion exchangers, amphoteric exchangers, or combinations thereof, where the cation exchangers can either be strongly acidic or weakly acidic, and the anion exchangers can be strongly basic or weakly basic. In an ion exchange treatment, the target ions in the HMO-containing stream are replaced in the stream by ions initially bound by the resin. Ion traffic is bidirectional, with the target ions fluxing onto the resin and the balancing ions moving out of the resin into the HMO-containing stream, thereby achieving electroneutrality. In ion adsorption, the target ions in the HMO-containing stream are removed from the stream and enriched on the surface of the resin. Ion traffic is mono-directional, with the process being analogous to the adsorption of molecules by a bed of activated carbon. The liquid itself is unchanged except for the removal of the target molecules. An example of ion adsorption would be the capture of acids by a weakly basic anion resin in free-base form, where the amine functional groups are neutral (not ionized), and not charged with counterions such as Cl⁻. Examples of such resins include Resindion Relite series RAM2, and Diaion WA series WA20. However, if the functional groups of that resin are charged and loaded with counter-ions they can be used in an ion exchange treatment.

### Cation Exchange

In an ion exchange treatment utilizing cation exchange, the stationary phase (resin) usually contains sulfonate groups. This cation exchange step removes positively charged components, e.g., residual ammonia, metal cations, and peptides. The binding capacity of the resins used are generally 1.2 to 2.2 eq/L. Typical resins used for the cation exchange include Dow Dowex 88, Resindion JC series JC603 and Resindion PK216.

### Anion Exchange

In an ion exchange utilizing anion exchange, the stationary phase (resin) is positively charged, and therefore retains negatively charged molecules by coulombic interaction. This step removes negatively charged components, e.g., sulphates, phosphates, organic acids, and negatively charged particles. The binding capacity of the resins used are generally 0.8 to 2.0 eq/L. Typical resins for anion exchange include Resindion Relite series such as D182, and JA100.

### Decolorization

A decolorization step can be performed to remove color-containing components. This step can be conducted using activated carbon, such as Norit CA1 activated carbon, a Hydrophobic Interaction Chromatography (HIC), or another adsorptive resin which can be functionalized, such as Resindion Relite RAD/F. The decolorization can be performed either before or after the anion exchange step.

### Concentration

A concentration step can be used to economically remove significant quantities of liquid from the HMO-containing stream using evaporation, reverse-osmosis filtration and nanofiltration. Evaporation processes can include, e.g., falling film evaporation, climbing film evaporation and rotary evaporation. The incoming solids concentration to the process is approximately 5 to 30 wt. %. The exit solids concentration from such a process is typically greater than 30 wt. %, preferably greater than 50 wt. %. More preferably, the solids concentration exiting the dewatering operation is 60 to 80 wt. %. The solids portion of the recovered material is greater than 80 wt. % HMO, with the remaining impurities primarily being alditols or di- or tri-saccharides.

### Heat Treatment

A heat treatment step can be used to kill bacteria or other undesired micro-organisms that may be present to any significant extent and is basically a pasteurization process. Any acceptable pasteurization conditions are possible, e.g., from 62.8° C. for 30 minutes to 72° C. for 20 seconds to 100° C. for 1-3 seconds.

### Indirect Drying

An indirect drying process is performed to increase the solids concentration of HMO to 90 wt. %+, while minimizing fines generation in the resultant solids stream. Preferably, the solids concentration of the HMO exiting the dryer is 95 wt. %+, more preferably 96 wt %+. For the purposes of this specification, indirect dryers include those devices that do not utilize direct contact of the material to be dried with a heated process gas for drying, but instead rely on heat transfer either through walls of the dryer, e.g., through the shell walls in the case of a drum dryer, or alternately through the walls of hollow paddles of a paddle dryer, as they rotate through the solids while the heat transfer medium circulates in the hollow interior of the paddles. Other examples of indirect dryers include contact dryers and vacuum drum dryers.

The heat transfer medium is preferably steam or heat transfer oils. More preferably, the heat transfer medium is steam. Indirect drying stands in sharp contrast to direct driers such as spray dryers, where a hot process gas moves through the vessel, directly contacting the circulating solids. Flash dryers or fluid bed dryers are additional examples of such direct drying methods.

### Drum Drying

Preferably, the indirect drying method is drum drying. In drum drying, the heated surface is the envelope of a rotating horizontal metal cylinder(s). The cylinder is preferably heated by steam condensing inside, bringing the temperature of the cylinder wall to 110-155° C. Steam pressure is maintained at about 2 to 5 bara to achieve these temperatures. Preferably, the cylinder wall of the drum dryer contacting the material to be dried is chrome plated. This prevents contamination of the dried product from metal components leaching from the unprotected walls of conventional drums, such as cast-iron drums; or corrosion products present on the drums from flaking off. Further, cast iron drums are not recommended for use at operating pH's less than 5, so chrome-plated drums offer much greater operating flexibility. Finally, it has unexpectedly been found that cast iron imparts a grey color to the HMO product, while chrome-plated drums do not.

Drum drying includes processes utilizing atmospheric double drum dryers, atmospheric single drum dryers, atmospheric twin drum dryers and enclosed drum dryers optionally operated under vacuum. Preferably, the drum drying is conducted in an atmospheric double drum dryer. Liquid feed can be applied to the surface of the rotating drum through nip feeding, roller feeding, dip feeding or spray and splash feeding. Preferably, the liquid feed is introduced to the double drum dryer with nip feeding. In nip feeding, the liquid feed is directed to the space between the adjacent counter rotating drums. A reservoir or pool of liquid builds between the drums, so that the “nip width” is the horizontal linear distance between the adjacent drums at the top surface of the pool. The higher this top surface relative to the centerline of the drums, the greater quantity of liquid in the pool. This quantity of liquid is also known as the “liquid holdup” because it represents additional residence time of the liquid prior to drying. The horizontal linear distance between the surfaces of the metal drums at their closest point is known as the drum gap.

The liquid pool that forms in the nip can begin to boil when exposed to the higher temperature of the drum walls. The counter rotation of the drums assists in drawing liquid into the pool of liquid in the nip and spreads it into a relatively thin film that is split between both rotating drums. Variables affecting drum dryer operation include rotation speed, drum gap size, nip width, drum temperature, steam pressure, feed temperature and feed solids concentration. Drum rotation speed is preferably 1 to 10 rpm. Drum gap size is preferably 0.1 mm to 2.0 mm, more preferably 0.1 to 0.3 mm. Nip width is preferably 0 to 50 mm, more preferably 0 to 10 mm. These ranges for the reduced nip width avoid crystallization/solidification in the nip with lump formation, which hamper achieving a steady operation. Feed temperature is preferably 4° C. to 110° C., more preferably, 4° C. to 95° C. or 50° C. to 110° C. Feed solids concentration is preferably 30 wt % to 70 wt %. Preferably, a homogenous feed is maintained over the full drum. More preferably, the homogenous feed is maintained with a feed header conduit having multiple conduit branches serving as feed points over the length of the drums. The feed points can comprise nozzles or other devices to direct the feed liquid. Feed pH is maintained between 3 to 7.5.

Roller feeding includes application rollers that rotate on the outside of the drum, where liquid feed is routed to a secondary nip formed between the applicator and the drum.

Dip coating includes those processes where the drums rotate through a reservoir of liquid, thereby adhering liquid to the wall of the drum.

In spray and splash feeding, liquid feed is directed upward onto the surface of the drums from below the drum.

The liquid feed on the surface of drum dries as the drum rotates, until the dried solids are eventually removed from the surface of the drum, e.g., with the assistance of a doctor blade or strings. The properties of the dried solids, e.g., the moisture content, morphology, and porosity are primarily adjusted by varying steam pressure, the width of the nip, feed properties, and the drum rotation speed, which affects the dryer residence time. Preferably, the HMO material has a residence time on the dryer of less than 3 minutes. Subsequently, milling and/or sieving step(s) may be used following the drying to obtain the desired particle size range.

### Milling

If a milling step is required, generally any milling method suitable for the type of solids and particle sizes targeted. Such milling equipment can include, e.g., ball mills, hammer mills, SAG mills, rod mills, Raymond mills and vertical mills.

For the purposes of this specification, unless otherwise specified, when referencing HMO recovered and purified utilizing the methods described in the specification, a “pure,” “purified” or “product” HMO stream has greater than 80% purity based on dry matter for a single HMO, or for mixtures of HMOs greater than 70% purity based on dry matter, for the combination; and a lactose content less than or equal to 10%, measured according to the procedures of Tables 1 & 2, and a moisture content of less than or equal to 10 wt %. The preferred properties of 2′-FL are summarized in Table 1. When the HMO stream has been treated to remove impurities but has not been completely dried, the term “pure,” “purified” or “product” refers to the material on a dry basis.

TABLE 1

| | Reference | |
|---|---|---|
| Analysis | Method | Maximum Levels |
| Appearance | Visual | white to off-white/ |
| (including color) | | ivory dry powder |
| Appearance in solution | Visual | Clear, colorless |
| (at 5%) | | to slightly yellow |
| KF titration | EP 2.5.12 v9 | <5% |
| pH (20° C., 5% solution) | EP 2.2.3 v9 | 3.0-7.5 |
| Total Ash (residue | FCC 11 | <0.5% w/w |
| after ashing) | appendix II | |

Heavy Metals

| | | |
|---|---|---|
| Mercury | EP 2.2.58 v9 | ≤0.1 mg/kg |
| Cadmium | EP 2.2.58 v9 | ≤0.05 mg/kg |
| Arsenic | EP 2.2.58 v9 | ≤0.2 mg/kg |
| Lead | EP 2.2.58 v9 | ≤0.05 mg/kg |
| Identification | Analytical Method | RT of main |
| | | component |
| | | corresponds to RT |
| | | of standard ±3% |

Carbohydrates

| | | |
|---|---|---|
| 2′-Fucosyllactose | HPAEC-PAD | ≥83% |
| Lactose | HPAEC-PAD | <8 area % |
| Difucosyllactose | HPAEC-PAD | <7 area % |
| 2-fucosyllactitol | HPAEC-PAD | <6% |
| Fucose | HPAEC-PAD | <6% |
| unknown 1 (likely sugar | HPAEC-PAD | |
| alcohol, RT 1.2) | | |
| unknown 2 (likely sugar | HPAEC-PAD | |
| alcohol, RT 1.4) | | |
| 2′-Fucosyl-D-lactulose | HPAEC-PAD | |
| Xylitol | HPAEC-PAD | |
| Dulcitol | HPAEC-PAD | |
| Mannitol | HPAEC-PAD | |
| Sorbitol/Galactitol | HPAEC-PAD | |
| Trihexose | HPAEC-PAD | |
| 3-Fucosylactose/ | HPAEC-PAD | |
| Fucosyl-Galactose | | |
| Glucose/Galactose | HPAEC-PAD | |
| GPE | HPAEC-PAD | |
| Fructose | HPAEC-PAD | |

The HPAEC-PAD method utilizes a pulsed amperometric detection technique. An exemplary HPAEC-PAD is illustrated in Tables 2-4.

TABLE 2
IEC-PAD Method Conditions

| | |
|---|---|
| Apparatus: | Dionex ICS-6000 AS Autosampler (AS-AP) |
| | Dionex ICS-6000 DC Column Compartment/ |
| | PAD Detector (DC) |
| | Dionex ICS-6000 Single Pump (SP) |
| Analytical | |
| Column: | |
| Type/Supplier/ | Thermo Fisher Scientific Dionex GM-4 Gradient |
| Part No.: | Mixer/CarboPac PA100 (250 × 4 mm) + CarboPac |
| | PA100 Guard Column (50 × 4 mm) |

TABLE 3
Dionex ™ ICS-6000 AS-AP Autosampler (AS-AP)

| | |
|---|---|
| Injection Volume: | 5 microliters (full loop) |
| Autosampler Tray Temperature | +5° C. |
| Setting: | |
| Autosampler Flush Solvent: | Deionized Water |
| Autosampler Flush Solvent Volume: | 250 microliters |
| Syringe Size: | 100 microliters |
| Syringe Speed: | 5 |
| Needle Height: | 2 mm |
| Tray Vibration: | On, 4 times before each injection |

Dionex ICS-6000 DC Column Compartment/PAD Detector (DC)

| | |
|---|---|
| Column Temperature: | 20° C. |
| Detector Type: | Pulsed Amperometric |
| | Detection (PAD) |
| Mode: | Integrated amperometric |
| Reference Electrode: | AgCl |
| Working Electrode: | Au on PTFE |
| Detector Waveform: | Gold, Carbo, Quad |
| Sampling Rate: | 2 Hz |

TABLE 4
Dionex ™ ICS-6000 Single Pump (SP)

| | |
|---|---|
| Flow Rate/Pressure: | 1.0 mL/min/−2600 psi |
| Method Gradient: | |
| Eluent A: | DI water |
| Eluent B: | 100 mM Sodium Hydroxide/ |
| | 75 mM Sodium Acetate |
| Eluent C: | 100 mM Sodium Hydroxide/ |
| | 150 mM Sodium Acetate |
| Eluent D: | 300 mM Sodium Hydroxide |

The HMO recovered and purified according to the methods described in this specification can be amorphous or crystalline. Preferably, the purity of the HMO on a dry basis is greater than 80 wt. % for a single HMO based on dry matter; or for mixtures of HMOs, greater than 70% purity based on dry matter, for the combination. More preferably, single HMO purity is greater than 90 wt. %.

Preferably, the HMO has at least one of the following characteristics: <2% lactulose, <3% fucose, <1% galactose, or <3% glucose.

The indirect drying of the claimed process results in a harsher thermal treatment of the HMO solids relative to direct drying processes, such as spray drying or flash drying. Unexpectedly, the HMOs resulting from the indirect drying process demonstrate excellent chemical resistance to degradation. The solids resulting from the indirect drying process can be in the form of granules, sheet material, flakes, or powder. Milling is an optional step that can subsequently be performed on this material to induce a desired particle size distribution in a more flexible and efficient manner than spray drying, where the particle size distribution is fixed by the spray drying operation. These advantages of indirect drying are in addition to its improved energy utilization and reduced risk of emissions and operator exposure to fines, relative to spray or flash drying. In addition, to avoid reduced levels of product yield, spray and flash drying typically require additional fines recycle-to-feed operations, which are not required for the described indirect drying process.

Unless otherwise specified, all particle sizes of the solid particles according to the present invention are determined by laser diffraction technique using a “Mastersizer 3000” of Malvern Instruments Ltd., UK. Further information on this particle size characterization method can e.g., be found in “Basic principles of particle size analytics,” Dr. Alan Rawle, Malvern Instruments Limited, Enigma Business Part, Grovewood Road, Malvern, Worcestershire, WR14 1XZ, UK and the “Manual of Malvern particle size analyzer.” Particular reference is made to the user manual number MAN 0096, Issue 1.0, November 1994. The particle size can be determined in the dry form, i.e., as powder or in suspension. Preferably, the particle size of the solid particles according to the present invention is determined as powder. The term d₅₀ (average particle size) means that particle diameter corresponding to 50% of the cumulative under size distribution by volume.

Preferably, the HMO has a fines fraction (less than or equal to 10 μm), of less than 10%, preferably less than 5%, more preferably less than 1%, most preferably less than 0.1%. The HMO also preferably has an average particle size (d₅₀), of greater than 100 μm, more preferably greater than 150 μm, even more preferably greater than 200 μm.

The HMO produced according to the claimed process demonstrates good flowability. Preferably, the HMO has a Carr index of less than 30, where the Carr index (C) is determined by the formula C=100(1−ρ_{B}/ρ_{T}), where ρ_{B} is the freely settled bulk density of the powder, and ρ_{T} is the tapped bulk density of the powder after “tapping down.” For free-flowing solids, the values of bulk and tapped density would be similar, so the value is small. For poorer flowing solids, the differences between these values would be larger, so that the Carr index would be larger.

Preferably, the HMO has a color in solution, measured by absorbance using a wavelength of 400 nm, of less than 0.3, more preferably less than 0.2, most preferably less than 0.1. Unless otherwise specified, for the purposes of this specification, the color measurement is obtained via the following procedure:

### Sample Solution

Prepare a 10% solution of the HMO in water, carefully homogenizing the solution. If the solution is still turbid after dissolution, it should be centrifuged or filtered before measurement. Transfer the clear solution to a 1 cm spectrophotometric cuvette and make sure to eliminate all the bubbles.

### Evaluation

Normalize the obtained absorbance value according to the formula:

⁴⁰⁰A=100×⁴⁰⁰A_{measured}/m

⁴⁰⁰A—normalized absorbance value at 400 nm
⁴⁰⁰A_{measured}—obtained absorbance value at 400 nm
m—weight of the sample in mg

Preferably, the HMO has a water content of less than 5 wt. %. In order to optimize product recovery, preferably, the HMO has a pH greater than 3.0, more preferably the HMO has a pH greater than 4.0. Typically, this is achieved by adjusting the pH of the HMO-containing stream to greater than 3.0 prior to the indirect drying step.

In one embodiment, the present disclosure provides a process for recovery and purification of HMOs comprising: (a) providing an HMO-containing fermentation broth comprising biomass; (b) separating the fermentation broth to form a separated HMO-containing stream and a biomass waste stream; (c) purifying the separated HMO-containing stream; (d) concentrating the separated HMO-containing stream; and (e) drying the product of steps (a)-(d) by an indirect drying method, thereby forming a purified HMO solid, wherein steps (c)-(d) can be performed in any order. Preferably, step (b) is performed with centrifugation, microfiltration, plate and frame filtration, recessed chamber filtration, belt filtration, vacuum filtration, horizontal metal leaf filtration, vertical metal-leaf filtration, stacked-disc filtration, rotary vacuum filtration and combinations thereof.

Purification step (c) can be selected from at least one of: (i) ultrafiltration; (ii) nanofiltration; (iii) deionization treatment; and (iv) decolorization, wherein sub-steps i-iv can be performed in any order. Deionization treatment step (iii) can be selected from ion adsorption or ion exchange. When a decolorization step is performed, it is preferably performed with at least one of activated carbon, a Hydrophobic Interaction Chromatography (HIC), and an adsorptive resin which can be functionalized, where the steps can be performed in any order. Preferably, step (d) is selected from at least one of evaporation, reverse-osmosis separation and nanofiltration, where the sub-steps can be performed in any order.

The following Examples further detail and explain the performance of the inventive HMO separation processes. Those skilled in the art will recognize many variations that are within the spirit of the invention and scope of the claims.

Examples 1 and 2 demonstrate the effects of high temperature degradation of HMOs.

### Example 1

Powdered 2′-fucosyllactose (“2′-FL”) commercially available as Aequival 2′-FL from Friesland Campina was mixed with water to form a 50 wt. % mixture. The mixture was spread onto three aluminum trays. One of the aluminum trays was heated at 80° C. for 129 minutes, a second was heated at 95° C. for 110 minutes and a third heated at 120° C. for 51 minutes. After heating, the 2′-FL concentration in each of the dried samples was measured by HPAEC-PAD. In addition, for each of the dried samples, the color of a 10% solution was measured at 438 nm by spectrophotometric measurement method. 2′-FL concentrations and color were similarly determined on three samples of the original Aequival 2′-FL powder. Results of the testing are shown in Table 5.

TABLE 5

| | 2′-FL Concentration | Color of 10% |
|---|---|---|
| | (mg/g) | solution @ 438 nm |
| Start powder #1 | 792 | 0.012 |
| Start powder #2 | 803 | 0.010 |
| Start powder #3 | 811 | n.d |
| 80° C., 129 minutes | 759 | 0.017 |
| 95° C., 110 minutes | 729 | 0.043 |
| 120° C., 51 minutes | 710 | 0.065 |

The data in Table 5 indicates that 2′-FL degradation occurs with higher drying temperatures. In addition, there is a corresponding increase in color with higher drying temperatures.

### Example 2

The 2′-FL powders dried and tested in Example 1 were analyzed using High Performance Anion-Exchange Chromatography to determine the changes in components of the solids because of the heating. These results are shown in Table 6.

TABLE 6

| | | | Powder | Powder | Powder |
|---|---|---|---|---|---|
| | | Original | heated at | heated at | heated at |
| | | Powder | 80° C. | 95° C. | 120° C. |
| Peak | Ret. Time | Rel. Time | Rel. Time | Rel. Time | Rel. Time |
| name | Min | % | % | % | % |
| Fucose | 2.09 | 0.08 | 0.63 | 1.52 | 1.90 |
| Glucose- | 3.74 | 0.56 | 1.13 | 1.59 | 1.69 |
| galactose | | | | | |
| Lactose | 6.92 | 1.18 | 1.31 | 1.14 | 0.94 |
| Lactulose | 7.16 | | 1.04 | 3.30 | 4.68 |
| 2′-FL | 8.48 | 96.05 | 93.90 | 90.47 | 88.80 |

The data of Table 6 indicates that along with the decrease in 2′-FL levels accompanying higher heating temperatures, sugars Fucose, Glucose-galactose, and Lactulose increase. Lactose trends down somewhat.

### Example 3

To evaluate the effect of increasing pH on 2′-FL samples dried using a drum dryer, two 1-Liter samples of 2′-FL were prepared as in Example 1 at a solids concentration of 50 wt. %. The pH of one of the samples was adjusted to 4.4 with caustic. The pH of the other sample was adjusted to 6.6 with caustic. Drum dryer testing was then performed in a drum dryer at a drum temperature of 110° C., operating at a rotation speed of 0.6 rpm. Dried samples were tested for 2′-FL concentration by HPLC. Results are shown in Table 7. The sample having the 4.4 pH was amorphous, and the sample having the 6.6 pH was crystalline, where crystallinity was measured by XRPD (X-ray Powder Diffraction).

TABLE 7

| | 2′-FL | Dry | 2′-FL on |
|---|---|---|---|
| | CONCENTRATION | matter | dry basis |
| | (MG/G) | (%) | (mg/g) |
| Starting | 803 | 95.84 | 838 |
| powder #1 | | | |
| Starting | 811 | n.d | n.d |
| powder #2 | | | |
| pH 4.4 | 794 | 96.70 | 821 |
| pH 6.6 | 800 | 96.34 | 830 |
| sample #1 | | | |
| pH 6.6 | 805 | 96.13 | 837 |
| sample #2 | | | |

The data in Table 7 illustrates that increasing pH improves 2′-FL concentration in the dried material.

### Example 4

A Brix 58 solution of 2′-FL was deposited at 50° C. and pH 4.5 with a feed rate of approximately 13 L/h on a drum dryer equipped with cast iron drums (drum diameter 500 mm/drum length 500 mm). The term “Brix” defines the sugar content in an aqueous solution. One degree Brix is 1 gram of sucrose in 100 grams of solution and represents the strength of the solution as percentage of mass. Although based on sucrose, the application to other sugars is performed in the same manner. The steam pressure was 3.2 bar (g), the gap setting between the drum was 0.17 mm, the rotational speed of the drums was 4.5 rpm, and the nip width was 50 mm. The product was collected as white flakes and dust at the knife, with a residual moisture of 2.13%. XRPD indicated the material was crystalline (form II).

### Example 5

A Brix 50 2′-FL solution was preheated to approximately 50° C. in a holding tank and subsequently transported by a pump to a chrome plated double drum drier (drum diameter 500 mm, length 500 mm) using a single hole feeding pipe. The steam pressure was 3.3 bar(g), the rotational speed of the drums was set to 1.2 rpm, and the gap setting is 0.2 mm as measured with product on the drums using a lead wire. The average nip width between the drums was 30 mm. During a capacity test of 30 minutes, the process was stable and a partial sheet of mostly flakes was achieved at the knife. The residual moisture content is on average 1.13%. The metal content of the 2′FL feed was analyzed with ICP-MS (Inductively Coupled Plasma-Mass Spectrometry). In a second test, the rotation speed was increased to 2.5 rpm. Further, a more concentrated 2′FL solution (Brix 58) was tested with the following conditions. The steam pressure was 3.0 bar(g) and the rotational speed 2.5 rpm (later in a follow up experiment increased to 3.5 rpm), a pool width of 50 mm and the gap setting was 0.15 mm as measured with product on the drums. Also, with these settings, the product was analyzed with respect to its metal content. Table 8 summarizes the leakage of iron and chrome into the dried 2′FL product, where the dry matter content varied between 1.1 and 2.4%.

TABLE 8

| Test | Fe (ppm) | Cr (ppm) |
|---|---|---|
| Feed (Brix 58) | 0.366 | 0.033 |
| Brix 50, 1.2 rpm | 0.994 | 0.0995 |
| Brix 50, 2.5 rpm | 0.83 | 0.092 |
| Brix 58 2.5 rpm | 0.781 | 0.067 |
| Brix 58 3.5 rpm | 0.79 | 0.053 |

The drum dried solids and the freeze-dried feed solution were also analyzed by HPAEC-PAD (Dionex ICS5000 with PAD detector, column CarboPac PA210 4×150 with 2×30 guard column, eluant A: 500 mM NaOH, Eluent B: water, Eluent C 100 mM NaOH). Selected peaks are presented in Table 9. No degradation upon drum drying is observed.

TABLE 9

| | | | drum dried | drum dried |
|---|---|---|---|---|
| | | | solid, | solid, |
| | HPAEC- | feed solution | 2.5 rpm | 1.2 rpm |
| | PAD | freeze dried | w % | w % |
| | Rt [min] | w % dry matter | dry matter | dry matter |
| Fucose | 2.36 | 0.03 | 0.03 | 0.03 |
| Galactose | 4.90 | 0.02 | 0.02 | 0.02 |
| Glucose | 5.33 | 0.01 | 0.01 | 0.01 |
| DFL | 9.64 | 1.75 | 1.79 | 1.81 |
| Lactose | 11.34 | 2.17 | 2.21 | 2.21 |
| Lactulose | 12.44 | 0.02 | 0.02 | 0.02 |

### Example 6

Six 5 kg 2′-FL Brix 58 samples were prepared, and the pH was adapted from 4.66 to various values using 4 M caustic solution or 4 M sulfuric acid. The range of pH tested varied between 5.85 and 4.03, while the feed solution was at ambient conditions (18-20° C.). The solution was transported from a holding tank to a double drum drier made from cast iron using a pump and distributed into the space between the drums using a single hole distribution pipe. The experiment was initiated with the following settings: 3.0 bar(g) steam pressure, 1.5 rpm rotational speed, 0.15 mm gap and a pool width of 30-35 mm. Product was collected from each test using a collection basket, and analyzed with ICP-MS. The iron content of the samples dropped from 66.9 ppm to 10.5 ppm, so a clear time effect could be observed. A second series of tests was performed using the same brix 58 2′FL solution, in which the rotational speed was varied. The pH (4.66) of the feed solution was not altered. The steam pressure was 3.2 bar(g), the drum speed was varied between 1.5 rpm and 9.5 rpm and the nip width was 30-35 mm. The liquid feed solution was fed between the drums, using a positive displacement pump. Table 10 illustrates the iron and chrome contents as measured in the product for each rpm setting, where the dry matter content of the dried solids varied between 1.3 and 2.4%.

TABLE 10

| Rotational speed | Fe (ppm) | Cr (ppm) |
|---|---|---|
| (rpm) | 0.34 | 0.09 |
| Feed analysis | (on dry matter) | (on dry matter) |
| 1.5 | 6.43 | 0.07 |
| 3.2 | 3.07 | 0.04 |
| 4.7 | 3.65 | 0.09 |
| 5.7 | 2.58 | 0.05 |
| 9.5 | 3.75 | 0.06 |

The results of Table 9 and 10 demonstrate that chrome plated drums are more resistant to metal leakage into the product than cast iron drums.

Examples 7 and 8 demonstrate the effects of indirect dryer construction (cast iron versus chrome plated) on product color.

### Example 7

A brix 58 solution of the mixture product containing both 2′-FL and Difucosyllactose (DFL) (14.5% DFL on total solid content) was diluted by adding deionized water to bring the concentration to brix 45. The solution pH is measured at pH 4.3, and then transported at ambient conditions by a pump to a double drum drier (cast iron, diameter 500 m, width 500 mm). The product was slightly viscous and transparent. The steam pressure during this test was set to 2.8 bar(g), the drum speed 5.1 rotations per minute, gap setting between the drums was 0.15 mm and the pool width was 10 mm maximum. A constant minimal feed resulted in a sheet at the knife. There was some discoloration of the drums. Product was scraped off the drums and collected in a collection basket. A second experiment was performed using the same operating conditions, but the liquid feed pH was adjusted from 4.3 to 5.2. Color results on the produced material are shown in Table 11. The dried solid from processing the pH 5.2 feed solution and the freeze-dried feed solution were also analyzed by HPAEC-PAD (Dionex ICS5000 with PAD detector, column CarboPac PA210 4×150 with 2×30 guard column, eluant A: 500 mM NaOH, Eluent B: water, Eluent C 100 mM NaOH). Selected peaks are presented in Table 12. No degradation upon drum drying is observed.

### Example 8

Solutions of 2′-FL and DFL were prepared as in Example 7, except that a chrome-plated drum was used. Two tests were performed. The first used a feed of Brix 58 at pH 4.3, and a second test was performed with a diluted feed of Brix 45 at the same pH of 4.3. The (cold, approx. 8° C.) product was deposited in the holding tank after which the liquid is diluted to the desired value if needed. Subsequently, the product was deposited in the pool using a Waukesha positive displacement pump and a feeding line with 8 mm holes. The gap was set to 0.15 mm (0.21 mm measured with product) and the steam pressure was 2.8 bar(g), and the rotational speed of the drums was 5.1 rpm. The product was collected in the collection basket and the color was measured. Color results on the produced material are shown in Table 11.

TABLE 11

| | ⁴⁰⁰A |
|---|---|
| 2′FL-DFL Drum drying cast iron pH 4.3 Brix 45 | 0.102 |
| 2′FL-DFL Drum drying cast iron pH 5.2 Brix 45 | 0.291 |
| 2′FL-DFL Drum drying chrome plated pH 4.3 Brix 45 | 0.009 |
| 2′FL-DFL Drum drying chrome plated pH 4.3 Brix 57 | 0.015 |

TABLE 12

| | | | Drum dried solid |
|---|---|---|---|
| | HPAEC- | feed solution | cast iron drum |
| | PAD | freeze-dried | pH 5.2, Brix 45 |
| | Rt [min] | w % dry matter | w % dry matter |
| Fucose | 2.36 | 0.00 | 0.01 |
| Galactose | 4.90 | 0.00 | 0.00 |
| Glucose | 5.33 | 0.04 | 0.03 |
| DFL | 9.64 | 14.64 | 14.57 |
| Lactose | 11.34 | 0.39 | 0.41 |
| Lactulose | 12.44 | 0.00 | 0.00 |

### Example 9

A feed solution of Lacto-N-tetraose (LNT) with Brix 33 was heated up to 50° C. and pumped using a displacement pump to a double drum drier (cast iron, dimensions diameter 500 mm, length 50 mm). The rotational speed was 1.5 rpm, the steam pressure was 3.2 barg and the gap setting between the drums was 0.10 mm. The nip width was approximately 50 mm. With these settings, s short test was made where the product formed a partially closed sheet at the knife. The residual moisture content of the product was 4.57%. The product was measured to be largely crystalline, consisting of 90% LNT form D and 10% LNT form B.

### Example 10

A solution of Lacto-N-neotetraose (LNnT) with Brix 40 was adjusted to 4.0 by adding sulfuric acid. Due to the slight acidity of the product, the tests were carried out on the double drum dryer with chrome-plated drums (diameter 500 mm, length 500 mm). The product was fed between the drums from the heated holding tank. The settings for this test are: 3.0 bar(g); rotational speed of 3.5 rpm; nip width of 40 mm. The process was stable with a partial sheet and flakes at the knife. The residual moisture is measured to be 6.1%. A second test was performed with the same set-up and settings, except that the rotational speed was decreased to 2.5 rpm to increase the dry matter content of the product. The lower rotational speed results in more flakes and powder at the knife. The residual moisture is approximately 5.5%.

The data of Table 10 demonstrates that chrome plated drums result in much less product color development relative to cast iron drums.

Other features, advantages and embodiments of the invention disclosed herein will be readily apparent to those exercising ordinary skill after reading the foregoing disclosure. In this regard, while specific embodiments of the invention have been described in considerable detail, variations and modifications of these embodiments can be affected without departing from the spirit and scope of the invention as described and claimed.

1. A process for recovery and purification of human milk oligosaccharides (HMOs) comprising:
(a) providing an HMO-containing fermentation broth comprising biomass;
(b) separating the fermentation broth to form a separated HMO-containing stream and a biomass waste stream;
(c) purifying the separated HMO-containing stream;
(d) concentrating the separated HMO-containing stream; and
(e) drying the product of steps (a)-(d) by an indirect drying method thereby forming a purified HMO,
wherein steps (c)-(d) can be performed in any order.

2. The process of claim 1, wherein step (b) is performed by centrifugation, microfiltration, plate and frame filtration, recessed chamber filtration, belt filtration, vacuum filtration, horizontal metal leaf filtration, vertical metal-leaf filtration, stacked-disc filtration, rotary vacuum filtration, or combinations thereof.

3. The process of claim 1, wherein step (c) is selected from the group consisting of:
(i) ultrafiltration;
(ii) nanofiltration;
(iii) deionization treatment; and
(iv) decolorization,
or a combination thereof, wherein sub-steps i-iv can be performed in any order.

4. The process of claim 3, wherein sub-step (iv) is performed with at least one method of activated carbon, a Hydrophobic Interaction Chromatography (HIC), and an adsorptive resin which can be functionalized, and wherein when the at least one method is more than one method, the methods are performed in any order.

5. The process of claim 1, wherein step (d) is selected from the group consisting of evaporation, reverse-osmosis filtration, and nanofiltration; or a combination thereof.

6. The process of claim 5, wherein the evaporation is selected from the group consisting of falling film evaporation, climbing film evaporation, and rotary evaporation; or a combination thereof.

7. The process of claim 1, further comprising a heat treatment step between steps (d) and (e).

8. The process of claim 2, wherein the microfiltration has a cut off in the range of 0.2 to 2.0 microns.

9. The process of claim 3, wherein the ultrafiltration utilizes a membrane with an MWCO of 1 kD to 300 kD.

10. The process of claim 3, wherein the nanofiltration utilizes a membrane with an MWCO of 200 dalton to 1000 dalton.

11. The process of claim 1, wherein the indirect drying method of step (e) is selected from the group consisting of drum drying, paddle drying, vacuum drum drying, and contact drying.

12. The process of claim 11, wherein the indirect drying method is drum drying.

13. The process of claim 12, wherein the drum drying has a dryer residence time of less than 3 minutes.

14. The process of claim 1, wherein the purified HMO is selected from the group consisting of 2′-fucosyllactose, 3-fucosyllactose, 2′,3-difucosyllactose, lacto-N-triose II, lacto-N-tetraose, lacto-N-neotetraose, lacto-N fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N difucohexaose II, 6′-galactosyllactose, 3′-galactosyllactose, lacto-N-hexaose and lacto-N neohexaose, sialyl-lacto-N-tetraose a, sialyllacto-N-tetraose b, sialyllacto-N-tetraose c, disialyllacto-N-tetraose, 3′-sialyllactose and 6′-sialyllactose; or mixtures thereof.

15. The process of claim 1, further comprising a milling step after the indirect drying method of step (e).

16. The process of claim 1, wherein the HMO-containing stream pH is adjusted to ≥4.0 prior to the indirect drying.

17. An HMO produced by indirect drying comprising at least one of:
(i) <2% lactulose;
(ii) <3% fucose;
(iii) <1% galactose; or
(iv) <3% glucose.

18. The HMO of claim 17 having a color in solution of less than 0.2.

19. The HMO of claim 17, wherein the indirect drying is conducted by a drum dryer at a drum temperature of at least 60° C.

20. A process for the production of HMOs comprising drying an HMO-containing stream in a drum dryer, the dryer comprising chrome-plated surfaces contacting the HMO-containing stream.

21. The process of claim 20, wherein the HMO has an average particle size (d₅₀) greater than 100 μm.

22. The process of claim 20, wherein the HMO has a Carr index of less than 30.

23. The process of claim 20, wherein the HMO has a fines fraction of less than 10%.

24.-25. (canceled)
